(19) United States
(12) Patent Application Publication
(10) Pub. No.: US 2023/0149088 A1
(43) Pub. Date: May 18, 2023

(54) LARGE VESSEL OCCLUSION DETECTION AND TREATMENT PREDICTION

(71) Applicant: Brown University, Providence, RI (US)

(72) Inventors: Harrison Bai, East Greenwich, RI (US); Ian Pan, Cleveland, OH (US); Thomas Yi, Providence, RI (US); Thi My Linh (Linh) Tran, Providence, RI (US)

(21) Appl. No.: 17/989,163

(22) Filed: Nov. 17, 2022

Related U.S. Application Data
(60) Provisional application No. 63/280,394, filed on Nov. 17, 2021.

(51) Int. Cl. A61B 34/10; G06T 7/00; G16H 50/20; G16H 30/40; G16H 50/50

(52) U.S. Cl. CPC A61B 34/10; G06T 7/0012; G16H 50/20; G16H 30/40; G16H 50/50; A61B 2034/105; A61B 2034/104; G06T 2207/30101; G06T 2207/10081; G06T 2207/20084

(57) ABSTRACT

Large vessel occlusion (LVO) detection and treatment prediction is provided via receiving a patient image depicting a medical scan of a patient; in response to identifying, via a first model, an LVO in the patient image, determining, via the first model whether the LVO is acute or chronic; in response to determining that the LVO is acute, determining via a second model different from the first model, a predicted treatment outcome for an immediate medical intervention for the LVO; and responsive to determining a successful treatment outcome for the LVO, assigning the immediate medical intervention to the patient.

### CROSS-REFERENCE TO RELATED APPLICATIONS

The present application claims priority to and the benefit of U.S. Provisional Application 63/280,394, filed Nov. 17, 2021, the contents of which are hereby incorporated in its entirety by reference.

### BACKGROUND

Stroke accounts for almost 5% of all disability-adjusted life-years and 10% of deaths worldwide. Large vessel occlusions (LVOs) account for about one-third of acute ischemic strokes but contribute to 90% of mortality and disabilities in these patients. Recent thrombectomy trials have shown that patients with LVOs can achieve better clinical outcomes with thrombectomy up to six hours from stroke onset and up to twenty-four hours for those who meet specific imaging criteria.

### SUMMARY

The present disclosure presents new and innovative systems and methods for detecting and predicting treatment outcomes for large vessel occlusions (LVOs). In one embodiment, a method is provided that includes receiving a patient image depicting a medical scan of a patient and determining, with a first model, whether the patient image depicts an LVO in the patient. Responsive to determining that the patient image depicts an LVO, the method may include determining, with a second model, a predicted treatment outcome for an immediate medical intervention for the LVO. Responsive to determining a successful treatment outcome, the method may include assigning the immediate medical intervention to the patient.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the disclosed subject matter.

### BRIEF DESCRIPTION OF THE FIGURES

FIG. 1 illustrates a system according to an exemplary embodiment of the present disclosure.

FIG. 2 illustrates a method according to an exemplary embodiment of the present disclosure.

FIG. 3 illustrates a latency comparison chart according to an exemplary embodiment of the present disclosure.

FIG. 4 illustrates a computer system according to an exemplary embodiment of the present disclosure.

### DETAILED DESCRIPTION OF EXAMPLE EMBODIMENTS

In practice, to activate the stroke code of an interventional team, a confirmation of a large vessel occlusion (LVO) by computed tomography angiography (CTA) must typically be met. CTA remains an accurate and effective vascular imaging modality to confirm or exclude the presence of LVOs. The Stroke Council of the American Heart Association recommends CTA in patients who might qualify for thrombectomy. CTA also has been regularly implemented into stroke imaging protocol at many primary stroke centers (PSCs) and comprehensive stroke centers (CSCs) to detect LVOs.

Because patients must typically be evaluated within the thrombectomy window to ensure successful recanalization, rapid detection of LVOs on CTAs is critical for patient triage, referral, and transfer prior to treatment. Given that LVOs are a time-sensitive emergency, LVO confirmation must therefore be done as quickly as possible to ensure positive patient outcomes in an expeditious manner. However, this task is typically subject to delays in hospital or similar settings due to infrastructure and expertise inconsistency. For example, certain smaller institutions may lack neuroradiology staffing or adequate teleradiology capabilities. Thus, CTA for patients with LVO may be overlooked and not be prioritized. For example, delays of ninety minutes or more from emergency department arrival to arterial access are not uncommon and may result in neurological deficits, increased mortality, and decreased chance for successful intervention. Therefore, there exists a need to quickly detect when arriving patients have LVOs that require medical intervention.

One solution to this problem is to use a combination of multiple models (e.g., artificial intelligence models, machine learning models, predictive models) to detect the presence of LVOs within medical scan images of the patients. When LVOs are detected, another model may be used to predict whether medical intervention is likely to succeed. If medical intervention is predicted to succeed, immediate medical intervention may be assigned to the patient, which may include alerting one or more medical professionals to the need for immediate medical care for the patient. In certain instances, detecting an LVO within the medical scan images may include determining whether the LVO was an acute LVO or is a chronic LVO. In such instances, the success of medical intervention may only be predicted for acute LVOs and chronic LVOs may be recommended for a different medical procedure (or different follow-up).

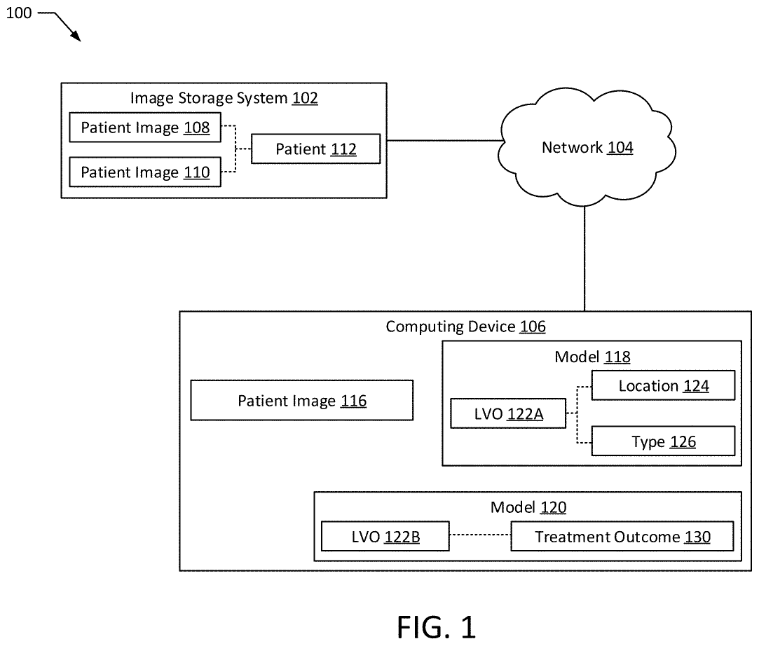

FIG. 1 illustrates a system 100 according to an exemplary embodiment of the present disclosure. The system 100 may be used at least in part to receive and analyze images of patients within a healthcare facility. For example, the system 100 may be implemented as part of a computing system of the healthcare facility. Such healthcare facilities may include, hospitals, emergency rooms, doctor's offices, and the like. The system 100 includes an image storage system 102, a network 104, and a computing device 106.

The image storage system 102 may be configured to store patient images 108, 110. The patient images 108, 110 may depict medical scans of the patient 112. For example, the image storage system 102 may be communicatively coupled to one or more patient imaging devices, such as magnetic resonance imaging (MRI) systems, computed tomography (CT) systems, and the like. The image storage system 102 may receive images of completed medical scans from the patient imaging devices. In certain implementations, the image storage system 102 may be implemented at least in part by a Picture Archiving and Communication System (PACS) of the healthcare site. The image storage system 102 may store the patient images 108, 110 in association with an indication of the patient 112 for which the medical scan was taken. Furthermore, the patient images 108, 110 may be stored with an indication of the type of medical scan depicted by the patient images 108, 110. For example, the indication may include a type of scan performed (e.g., an MRI scan, a CT scan), an indication of the portion of the patient that was scanned (e.g., heart, head, torso), and the like.

The computing device 106 may be configured to receive patient images 116 from the image storage system 102, institute analysis of the patient images 116 for LVOs within corresponding patients, and to determine whether the detected LVOs require immediate medical intervention. In certain instances, the computing device 106 may be configured to receive patient images 116 from the image storage system 102 via a network 104. For example, when a new patient image 108, 110 is received by the image storage system 102, the image storage system 102 may transmit a notification to the computing device 106. The computing device 106 and/or the image storage system 102 may determine whether the newly-received patient image 108, 110 needs to be analyzed for potential LVOs. For example, the image storage system 102 and/or the computing device 106 may determine whether the medical scan depicted by the patient image 108, 110 corresponds to an area of the patient's 112 body in which LVOs can be detected. For example, LVOs may be detectable within CT angiogram scans of a patient's 112 head or neck, and the image storage system 102 and/or the computing device 106 may be configured to determine whether a newly-received patient image 108, 110 depicts a CT angiogram scan of a head or neck. For patient images 108, 110 in which LVOs may be detectable, the image storage system 102 may transmit a copy of the patient image 108, 110 the computing device 106. Additionally or alternatively, the computing device 106 may retrieve a copy of the patient image from the image storage system 102.

The computing device 106 may then analyze the patient image 116 with models 118, 120. For example, the computing device 106 may analyze the patient image 116 with a first model 118 to determine whether the patient image 116 depicts an LVO 122A-B (generally or collectively, LVO 122) within the patient. If the patient image 116 depicts an LVO 122A, a second model 120 may analyze the patient image 116 to predict a treatment outcome 130 for the LVO 122B. The models 118, 120 may be implemented as one or more predictive models. For example, the models 118, 120 may be implemented as machine learning models, such as convolutional neural networks (CNNs). In one specific example, the model 118 and the model 120 may both be implemented as deep learning convolutional neural networks.

The model 118, as described above, may be configured to determine whether the patient image 116 depicts an LVO 122. For example, the model 118 may be trained to analyze one or more aspects of the patient image 116 (e.g., higher density regions, locations of higher density regions within the patient image and/or within a particular patient) to determine whether the patient image 116 depicts an LVO 122. If the patient image 116 depicts an LVO 122, the model 118 may determine a location 124 for the LVO 122 (e.g., a location within the patient image 116 answers or location within the patient 112). Additionally or alternatively, the model 118 may determine a type 126 for the LVO 122. For example, the model 118 may be trained to determine whether the LVO 122 is an acute LVO requiring quicker medical intervention or a chronic LVO that may be successfully treated with less immediate medical intervention.

For patient images 116 depicting an LVO 122, the model 120 may be configured to analyze the LVO 122 and/or the patient image 116 to predict a treatment outcome 134 based on immediate medical intervention for the LVO 122. For example, the treatment outcome 130 may be determined to indicate the likelihood of success for an immediate medical intervention procedure, such as a thrombectomy (e.g., endovascular therapy of mechanical thrombectomy). In certain instances, the treatment outcome 130 may indicate whether the immediate medical intervention is likely to succeed or is not likely to succeed. For example, the treatment outcome 130 may be determined as a predicted Thrombolysis in Cerebral Infarction (TICI) score and/or as a predicted Modified Rankin Score (mRS) for a thrombectomy procedure.

The models 118, 120 may be trained prior to use by the computing device 106. For example, the model 118 may be trained to detect the presence of LVOs within patient images by providing the model 118 with anonymized, raw images of medical scans (e.g., CT angiogram scans), a subset of which may contain LVOs. The images may be tagged (e.g., in metadata for the images) with an indication of whether the image depicts an LVO and, in certain implementations, a type of LVO depicted (e.g., an acute LVO, a chronic LVO). As another example, the model 120 may be trained by providing the model 120 with images containing acute LVOs and corresponding TICI scores (e.g., retrieved from a report for a thrombectomy procedure to treat the depicted acute LVO) and/or corresponding mRS scores (e.g., retrieved from longer-term medical records, such as electronic medical records, prepared by a neurologist after a thrombectomy procedure). Based on the tagged training data described herein, one or more aspects of the model 118, 120 (e.g., features analyzed, feature weights) may be added, removed, or adjusted (e.g., weighted higher, weighted lower).

As depicted, the image storage system 102 and the computing device 106 communicate via the network 104. The computing device 106 and/or the image storage system 102 may communicate with the network 104 using one or more wired network interfaces (e.g., Ethernet interfaces) and/or wireless network interfaces (e.g., Wi-Fi®, Bluetooth®, and/or cellular data interfaces). In certain instances, the network 104 may be implemented as a local network (e.g., a local area network), a virtual private network, L1, and/or a global network (e.g., the Internet).

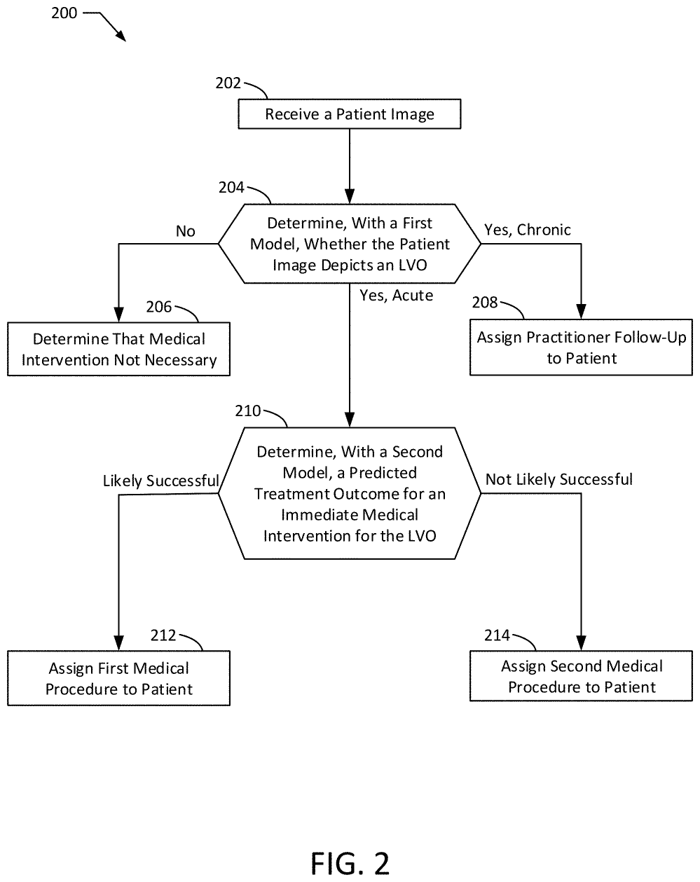

FIG. 2 illustrates a method 200 according to an exemplary embodiment of the present disclosure. The method 200 may be performed to analyze incoming patient images for potential LVOs and to assign corresponding medical procedures based on the detected LVOs. The method 200 may be implemented on a computer system, such as the system 100 discussed in greater detail in relation to FIG. 1. For example, the method 200 may be implemented by the computing device 106. The method 200 may also be implemented by a set of instructions stored on a computer-readable medium that, when executed by a processor, cause the computer system to perform the method 200. For example, all or part of the method 200 may be implemented by a processor and a memory of the computing device 106. Although the examples provided herein are described with reference to the flowchart illustrated in FIG. 2, many other methods of performing the acts associated with FIG. 2 may be used. For example, the order of some of the blocks may be changed, certain blocks may be combined with other blocks, one or more of the blocks may be repeated, and some of the blocks described may be optional.

The method 200 may begin with receiving a patient image (block 202). For example, a computing device 106 may receive a patient image 116 from an image storage system 102, such as a PACS image storage system for a hospital. The patient image 116 may depict a medical scan of an associated patient 112, such as a CTA scan of a head or neck of a patient 112.

The computing device 106 may then determine, with a first model 118, whether the patient image 116 depicts an LVO 122 (block 204). For example, the model 118 may analyze the patient image 116 for high density sections within the patient image 116) to determine whether the patient image 116 depicts an LVO 122 within the patient 112. If an LVO 122 is detected, the model 118 may also determine a location 124 and/or a type 126 of the LVO 122. For example, in certain instances, in addition to detecting the LVO 122, the model 118 may be configured (e.g., trained) to distinguish between acute LVOs and chronic LVOs. In such instances, the model 118 may further determine whether the patient image 116 depicts an acute LVO or depicts a chronic LVO.

If the patient image 116 does not depict an LVO 122, it may be determined that medical intervention is not necessary (block 206). For example, the computing device 106 may determine that medical intervention to treat an LVO 122 is not needed for a patient 112 corresponding to the patient image 116. Accordingly, the computing device 106 may store an indication (e.g., in an electronic medical record for the patient 112 and/or in a computing system for the healthcare facility) that the patient image 116 did not depict an LVO 122. In certain instances, the patient image 116 may still be flagged for manual review (e.g., by a radiologist or other doctor).

If the patient image 116 depicts a chronic LVO 122, practitioner follow-up may be assigned to the patient 112 (block 208). For example, the computing device 106 may determine that, although an LVO 122 was detected within the patient 112, because the LVO 122 was a chronic LVO, immediate medical intervention is not needed. Accordingly, practitioner follow-up may be assigned to the patient 112 (e.g., within an electronic medical record for the patient 112 or in a computing system for the healthcare facility). In certain instances, practitioner follow-up may include a follow-up consultation with, for example, a neurologist or other doctor. The practitioner follow-up may be recommended or assigned to take place within a longer time period (e.g., 1-4 weeks) than treatment for an acute LVO 122.

If the patient image 116 depicts an acute LVO 122, the computing device 106 may use a second model 120 to determine a predicted treatment outcome 130 for an immediate medical intervention for the LVO 122 (block 210). For example, the second model 120 may predict a treatment outcome 130 for an immediate medical intervention (e.g., a thrombectomy) to treat the detected LVO 122. The treatment outcome 130 may include a predicted likelihood of immediate success for the medical intervention, such as a predicted TICI score for the medical intervention. Additionally or alternatively, the treatment outcome 130 may include a predicted likelihood of long-term success (e.g., patient improvement) as a result of the medical intervention, such as a predicted mRS score for the patient 112 after undergoing the medical intervention.

If it is determined that the predicted treatment outcome 130 is likely successful, a first medical procedure may be assigned to the patient 112 (block 212). For example, the computing device 106 may determine that the predicted treatment outcome 130 is likely successful if the treatment outcome 130 exceeds a predetermined threshold. In one specific example, it may be determined that the predicted treatment outcome 130 is likely successful if one or both of the predicted TICI score and the predicted mRS score exceed a predetermined threshold. In such instances, a first medical procedure (e.g., a thrombectomy) may be assigned to the patient 112. For example, based on the determination that immediate medical intervention is likely to succeed, the computing device 106 may assign the first medical procedure to electronic medical record associated with the patient 112. Given the need for rapid medical intervention, the first medical procedure may be recommended or assigned to take place immediately or within a shorter time period (e.g., within one to four hours) than for treatment of a chronic LVO 122.

If it is determined that the predicted treatment outcome 130 is not likely successful, a second medical procedure may be assigned to the patient 112 (block 214). For example, the computing device 106 may determine that the predicted treatment outcome 130 is not likely successful if the predicted outcome 130 does not exceed a predetermined threshold (e.g., a predetermined threshold as discussed above in connection with block 212). In such instances, a second medical procedure (e.g., intravenous tissue-type plasminogen activator (IV tPA)) may be assigned to the patient 112. In certain implementations, rather than a specific medical procedure, further practitioner follow-up may be assigned to the patient 112, similar to the practitioner follow-up assigned at block 208. The second medical procedure (or practitioner follow-up) may be assigned to take place within a medium time period (e.g., within three hours to one week) between the treatment times for chronic LVOs and acute LVOs determine to have likely successful treatment via thrombectomy.

Certain implementations of the method 200 may differ from the specific implementations discussed above. For example, it should be understood that, in certain implementations, the model 118 may not be configured to distinguish between chronic LVOs and acute LVOs. In such instances, any detection of an LVO 122 may result in further processing as an acute LVO (e.g., by proceeding from block 204 to block 210). As another example, rather than specifically assigning medical procedures to the patient, physicians or surgeons responsible for performing or validating such procedures may be notified (e.g., using electronic notification channels such as email, messaging platforms, notifications, and the like).

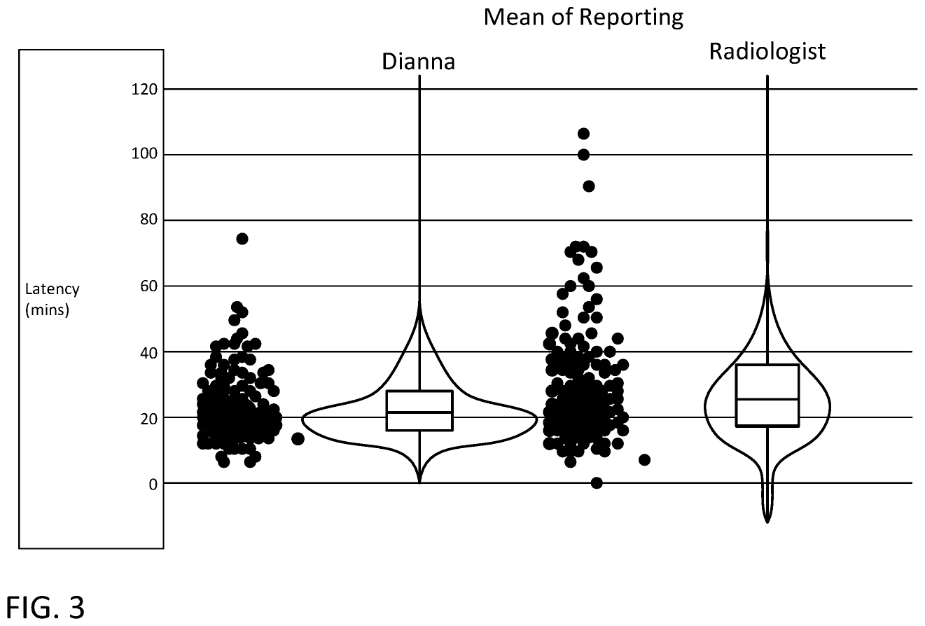

The method 200 may allow for improved and automated analysis of patient images 116 that potentially depict LVOs 122 within patients 112. For example, the method 200 may allow for dramatically reduced latencies between when an initial scan is performed of a patient 112 and when an initial determination is made for whether immediate medical intervention is necessary. For example, FIG. 3 illustrates a latency comparison chart 300 according to an exemplary embodiment of the present disclosure. The chart 300 depicts reporting time latencies 306, 308 for automated systems using techniques similar to those discussed above (labeled as DIANA in the chart 300) and reporting time latencies 310, 312 for conventional techniques relying on radiologist evaluation of patient images 116. The mean latency using the method 200 and the above-discussed techniques is 27.57 minutes ±18.09 minutes with a median latency of 23.93 minutes, compared to latencies of 41.11 minutes ±71.39 minutes and a median latency of 28.28 minutes for conventional techniques. Accordingly, the present-discussed techniques reduce the overall time necessary to determine that a patient 112 requires treatment for an LVO 122 and to begin immediate medical intervention for the treatment as necessary. These techniques therefore increase the effectiveness of medical intervention, reducing overall mortality from LVOs 122 and saving lives.

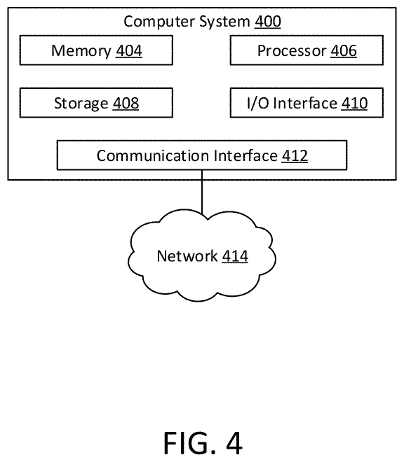

FIG. 4 illustrates an example computer system 400 that may be used to implement one or more of the devices and/or components discussed herein, such as the computing device 106 and/or the image storage system 102 discussed in greater detail in regard to FIG. 1. In various embodiments, one or more computer systems 400 perform one or more steps of one or more methods described or illustrated herein, such as the method 200 discussed in greater detail in regard to FIG. 2. In some embodiments, one or more computer systems 400 provide the functionalities described or illustrated herein. In some embodiments, software running on one or more computer systems 400 performs one or more steps of one or more methods described or illustrated herein or provides the functionalities described or illustrated herein. Some embodiments include one or more portions of one or more computer systems 400. Herein, a reference to a computer system may encompass a computing device, and vice versa, where appropriate. Moreover, a reference to a computer system may encompass one or more computer systems, where appropriate.

This disclosure contemplates any suitable number of computer systems 400. This disclosure contemplates the computer system 400 taking any suitable physical form. As example and not by way of limitation, the computer system 400 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the computer system 400 may include one or more computer systems 400; be unitary or distributed; span multiple locations; span multiple machines; span multiple data centers; or reside in a cloud, which may include one or more cloud components in one or more networks. Where appropriate, one or more computer systems 400 may perform without substantial spatial or temporal limitation one or more steps of one or more methods described or illustrated herein. As an example and not by way of limitation, one or more computer systems 400 may perform in real time or in batch mode one or more steps of one or more methods described or illustrated herein. One or more computer systems 400 may perform at different times or at different locations one or more steps of one or more methods described or illustrated herein, where appropriate.

In some embodiments, computer system 400 includes a processor 406, memory 404, storage 408, an input/output (I/O) interface 410, and a communication interface 412. Although this disclosure describes and illustrates a computer system having a given number of various components in a given arrangement, this disclosure contemplates any suitable computer system having any suitable number of any suitable components in any suitable arrangement.

In some embodiments, the processor 406 includes hardware for executing instructions, such as those making up a computer program. As an example and not by way of limitation, to execute instructions, the processor 406 may retrieve (or fetch) the instructions from an internal register, an internal cache, memory 404, or storage 408; decode and execute the instructions; and then write one or more results to an internal register, internal cache, memory 404, or storage 408. In various embodiments, the processor 406 may include one or more internal caches for data, instructions, or addresses. This disclosure contemplates the processor 406 including any suitable number of any suitable internal caches, where appropriate. As an example and not by way of limitation, the processor 406 may include one or more instruction caches, one or more data caches, and one or more translation lookaside buffers (TLBs). Instructions in the instruction caches may be copies of instructions in memory 404 or storage 408, and the instruction caches may speed up retrieval of those instructions by the processor 406. Data in the data caches may be copies of data in memory 404 or storage 408 that are to be operated on by computer instructions; the results of previous instructions executed by the processor 406 that are accessible to subsequent instructions or for writing to memory 404 or storage 408; or any other suitable data. The data caches may speed up read or write operations by the processor 406. The TLBs may speed up virtual-address translation for the processor 406. In various embodiments, processor 406 may include one or more internal registers for data, instructions, or addresses. This disclosure contemplates the processor 406 including any suitable number of any suitable internal registers, where appropriate. Where appropriate, the processor 406 may include one or more arithmetic logic units (ALUs), be a multi-core processor, or include one or more processors 406. Although this disclosure describes and illustrates a given processor, this disclosure contemplates any suitable processor.

In some embodiments, the memory 404 includes main memory for storing instructions for the processor 406 to execute or data for processor 406 to operate on. As an example, and not by way of limitation, computer system 400 may load instructions from storage 408 or another source (such as another computer system 400) to the memory 404. The processor 406 may then load the instructions from the memory 404 to an internal register or internal cache. To execute the instructions, the processor 406 may retrieve the instructions from the internal register or internal cache and decode them. During or after execution of the instructions, the processor 406 may write one or more results (which may be intermediate or final results) to the internal register or internal cache. The processor 406 may then write one or more of those results to the memory 404. In some embodiments, the processor 406 executes only instructions in one or more internal registers or internal caches or in memory 404 (as opposed to storage 408 or elsewhere) and operates only on data in one or more internal registers or internal caches or in memory 404 (as opposed to storage 408 or elsewhere). One or more memory buses (which may each include an address bus and a data bus) may couple the processor 406 to the memory 404. The bus may include one or more memory buses, as described in further detail herein. In various embodiments, one or more memory management units (MMUs) reside between the processor 406 and memory 404 and facilitate accesses to the memory 404 requested by the processor 406. In various embodiments, the memory 404 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 404 may include one or more memories 404, where appropriate. Although this disclosure describes and illustrates given memory implementations, this disclosure contemplates any suitable memory implementation.

In some embodiments, the storage 408 includes mass storage for data or instructions. As an example and not by way of limitation, the storage 408 may include a hard disk drive (HDD), a floppy disk drive, flash memory, an optical disc, a magneto-optical disc, magnetic tape, or a Universal Serial Bus (USB) drive or a combination of two or more of these. The storage 408 may include removable or non-removable (or fixed) media, where appropriate. The storage 408 may be internal or external to computer system 400, where appropriate. In various embodiments, the storage 408 is non-volatile, solid-state memory. In various embodiments, the storage 408 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 408 taking any suitable physical form. The storage 408 may include one or more storage control units facilitating communication between processor 406 and storage 408, where appropriate. Where appropriate, the storage 408 may include one or more storages 408. Although this disclosure describes and illustrates given storage, this disclosure contemplates any suitable storage.

In some embodiments, the I/O Interface 410 includes hardware, software, or both, providing one or more interfaces for communication between computer system 400 and one or more I/O devices. The computer system 400 may include one or more of these I/O devices, where appropriate. One or more of these I/O devices may enable communication between a person (i.e., a user) and computer system 400. As an example and not by way of limitation, an I/O device may include a keyboard, keypad, microphone, monitor, screen, display panel, mouse, printer, scanner, speaker, still camera, stylus, tablet, touch screen, trackball, video camera, another suitable I/O device or a combination of two or more of these. An I/O device may include one or more sensors. Where appropriate, the I/O Interface 410 may include one or more device or software drivers enabling processor 406 to drive one or more of these I/O devices. The I/O interface 410 may include one or more I/O interfaces 410, where appropriate. Although this disclosure describes and illustrates a given I/O interface, this disclosure contemplates any suitable I/O interface or combination of I/O interfaces.

In some embodiments, communication interface 412 includes hardware, software, or both providing one or more interfaces for communication (such as, for example, packet-based communication) between computer system 400 and one or more other computer systems 400 or one or more networks 414. As an example and not by way of limitation, communication interface 412 may include a network interface controller (NIC) or network adapter for communicating with an Ethernet or any other wire-based network or a wireless NIC (WNIC) or wireless adapter for communicating with a wireless network, such as a Wi-Fi network. This disclosure contemplates any suitable network 414 and any suitable communication interface 412 for the network 414. As an example and not by way of limitation, the network 414 may include one or more of an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 may communicate with a wireless PAN (WPAN) (such as, for example, a Bluetooth® WPAN), a WI-FI network, a WI-MAX network, a cellular telephone network (such as, for example, a Global System for Mobile Communications (GSM) network), or any other suitable wireless network or a combination of two or more of these. Computer system 400 may include any suitable communication interface 412 for any of these networks, where appropriate. Communication interface 412 may include one or more communication interfaces 412, where appropriate. Although this disclosure describes and illustrates a given communication interface implementations, this disclosure contemplates any suitable communication interface implementation.

The computer system 402 may also include a bus. The bus may include hardware, software, or both and may communicatively couple the components of the computer system 400 to each other. As an example and not by way of limitation, the bus may include an Accelerated Graphics Port (AGP) or any other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local bus (VLB), or another suitable bus or a combination of two or more of these buses. The bus may include one or more buses, where appropriate. Although this disclosure describes and illustrates a given bus, this disclosure contemplates any suitable bus or interconnect.

Herein, a computer-readable non-transitory storage medium or media may include one or more semiconductor-based or other types of integrated circuits (ICs) (e.g., field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including various components, elements, features, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a certain function encompasses that apparatus, system, component, whether or not it or that certain function is activated, turned on, or unlocked, as long as that apparatus, system, or component is so adapted, arranged, capable, configured, enabled, operable, or operative. Additionally, although this disclosure describes or illustrates various embodiments as providing various advantages, various embodiments may provide none, some, or all of these advantages.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A method, comprising
receiving a patient image depicting a medical scan of a patient;
in response to identifying, via a first model, a large vessel occlusion (LVO) in the patient image, determining, via the first model whether the LVO is acute or chronic;
in response to determining that the LVO is acute, determining via a second model different from the first model, a predicted treatment outcome for an immediate medical intervention for the LVO; and
responsive to determining a successful treatment outcome for the LVO, assigning the immediate medical intervention to the patient.

2. The method of claim 1, further comprising:
receiving a second patient image depicting a second medical scan of a second patient;
in response to identifying, via the first model, a second LVO in the patient image, determining, via the first model whether the second LVO is acute or chronic;
in response to determining that the second LVO is acute, determining via the second model, a second predicted treatment outcome for the immediate medical intervention for the second LVO; and
responsive to determining an unsuccessful treatment outcome for the second LVO, assigning a second medical intervention to the patient, different from the immediate medical intervention.

3. The method of claim 2, wherein the immediate medical intervention is a thrombectomy performed within one to four hours of receiving the patient image and the second medical intervention is an intravenous tissue-type plasminogen activator procedure performed within three hours to one week of receiving the second patient image.

4. The method of claim 1, further comprising:
receiving a second patient image depicting a second medical scan of a second patient;
in response to identifying, via the first model, a second LVO in the patient image, determining, via the first model whether the second LVO is acute or chronic; and
in response to determining that the second LVO is chronic, generating a follow-up for the patient with a practitioner.

5. The method of claim 1, further comprising:
receiving a second patient image depicting a second medical scan of a second patient; and
in response to determining, via the first model, that the second patient image does not depict a second LVO in the second patient, flagging the second medical scan for manual review.

6. The method of claim 1, wherein the predicted treatment outcome for athern immediate medical intervention is predicted according to at least one of a Thrombolysis in Cerebral Infarction (TICI) score and a predicted Modified Rankin Score (mRS) for a thrombectomy procedure.

7. The method of claim 1, wherein the patient image is a computed tomography angiography (CTA) image.

8. A system, comprising:
a processor; and
a memory including instructions that when executed by the processor perform operations comprising:
receiving a patient image depicting a medical scan of a patient;
in response to identifying, via a first model, a large vessel occlusion (LVO) in the patient image, determining, via the first model whether the LVO is acute or chronic;
in response to determining that the LVO is acute, determining via a second model different from the first model, a predicted treatment outcome for an immediate medical intervention for the LVO; and
responsive to determining a successful treatment outcome for the LVO, assigning the immediate medical intervention to the patient.

9. The system of claim 8, the operations further comprising:
receiving a second patient image depicting a second medical scan of a second patient;
in response to identifying, via the first model, a second LVO in the patient image, determining, via the first model whether the second LVO is acute or chronic;
in response to determining that the second LVO is acute, determining via the second model, a second predicted treatment outcome for the immediate medical intervention for the second LVO; and
responsive to determining an unsuccessful treatment outcome for the second LVO, assigning a second medical intervention to the patient, different from the immediate medical intervention.

10. The system of claim 9, wherein the immediate medical intervention is a thrombectomy performed within one to four hours of receiving the patient image and the second medical intervention is an intravenous tissue-type plasminogen activator procedure performed within three hours to one week of receiving the second patient image.

11. The system of claim 8, the operations further comprising:
receiving a second patient image depicting a second medical scan of a second patient;
in response to identifying, via the first model, a second LVO in the patient image, determining, via the first model whether the second LVO is acute or chronic; and
in response to determining that the second LVO is chronic, generating a follow-up for the patient with a practitioner.

12. The system of claim 8, the operations further comprising:
receiving a second patient image depicting a second medical scan of a second patient; and
in response to determining, via the first model, that the second patient image does not depict a second LVO in the second patient, flagging the second medical scan for manual review.

13. The system of claim 8, wherein the predicted treatment outcome for the immediate medical intervention is predicted according to at least one of a Thrombolysis in Cerebral Infarction (TICI) score and a predicted Modified Rankin Score (mRS) for a thrombectomy procedure.

14. The method of claim 1, wherein the patient image is a computed tomography angiography (CTA) image.

15. A memory device including instructions that when executed by a processor perform operations including:
receiving a patient image depicting a medical scan of a patient;
in response to identifying, via a first model, a large vessel occlusion (LVO) in the patient image, determining, via the first model whether the LVO is acute or chronic;
in response to determining that the LVO is acute, determining via a second model different from the first model, a predicted treatment outcome for an immediate medical intervention for the LVO; and
responsive to determining a successful treatment outcome for the LVO, assigning the immediate medical intervention to the patient.

16. The memory device of claim 15, the operations further comprising:
receiving a second patient image depicting a second medical scan of a second patient;
in response to identifying, via the first model, a second LVO in the patient image, determining, via the first model whether the second LVO is acute or chronic;
in response to determining that the second LVO is acute, determining via the second model, a second predicted treatment outcome for the immediate medical intervention for the second LVO; and
responsive to determining an unsuccessful treatment outcome for the second LVO, assigning a second medical intervention to the patient, different from the immediate medical intervention.

17. The memory device of claim 16, wherein the immediate medical intervention is a thrombectomy performed within one to four hours of receiving the patient image and the second medical intervention is an intravenous tissue-type plasminogen activator procedure performed within three hours to one week of receiving the second patient image.

18. The memory device of claim 15, the operations further comprising:
receiving a second patient image depicting a second medical scan of a second patient;
in response to identifying, via the first model, a second LVO in the patient image, determining, via the first model whether the second LVO is acute or chronic; and
in response to determining that the second LVO is chronic, generating a follow-up for the patient with a practitioner.

19. The memory device of claim 15, the operations further comprising:
receiving a second patient image depicting a second medical scan of a second patient; and
in response to determining, via the first model, that the second patient image does not depict a second LVO in the second patient, flagging the second medical scan for manual review.

20. The memory device of claim 15, wherein the predicted treatment outcome for the immediate medical intervention is predicted according to at least one of a Thrombolysis in Cerebral Infarction (TICI) score and a predicted Modified Rankin Score (mRS) for a thrombectomy procedure.
